SEQUENCE LISTING (1 sequence)

SEQ ID NO: 1 - protein, 960 aa, Arabidopsis thaliana
MTSMIVKTTPELFKGNGVFRTDHLGENRMVSRSNRLGDGSNRFPRTGTIHCQRLSIAKTGLHRETKARAILSPVSDPAASIAQKLGGKGANLAEMASIGLSVPPGLTISTEACQQYQIAGKKLPEGLWEEILEGLSFIERDIGASLADPSKPLLLSVRSGAAISMPGMMDTVLNLGLNDQVVVGLAAKSGERFAYDSFRRFLDMFGDVVMGIPHAKFEEKLERMKERKGVKNDTDLSAADLKELVEQYKSVYLEAKGQEFPSDPKKQLELAIEAVFDSWDSPRANKYRSINQITGLKGTAVNIQCMVFGNMGDTSGTGVLFTRNPSTGEKKLYGEFLVNAQVWHLSQCVNLISTRIRTPEDLDTMKRFMPEAYAELVENCNILERHYKDMMDIEFTVQEERLWMLQCRAGKRTGKGAVKIAVDMVGEGLVEKSSAIKMVEPQHLDQLLHPQFHDPSGYREKVVAKGLPASPGAAVGQVVFTAEEAEAWHSQGKTVILVRTETSPDDVGGMHAAEGILTARGGMTSHAAVVARGWGKCCIAGCSEIRVDENHKVLLIGDLTINEGEWISMNGSTGEVILGKQALAPPALSPDLETFMSWADAIRRLKVMANADTPEDAIAARKNGAQGIGLCRTEHMIVCIQMFNVVFGLVFKFFGADRIKAVRKMIMAVTTEQRKASLDILLPYQRSDFEGIFRAMDGLPVTIRLLDPPLHEFLPEGDLDNIVHELAEETGVKEDEVLSRIEKLSEVNPMLGFRGCRLGISYPELTEMQARAIFEAAASMQDQGVTVIPEIMVPLVGTPQELGHQVDVIRKVAKKVFAEKGHTVSYKVGTMIEIPRAALIADEIAKEAEFFSFGTNDLTQMTFGYSRDDVGKFLPIYLAKGILQHDPFEVLDQQGVGQLIKMATEKGRAARPSLKVGICGEHGGDPSSVGFFAEAGLDYVSCSPFRVPIARLAAAQVVVA

(19) United States
(12) Patent Application Publication
(10) Pub. No.: US 2002/0106712 A1
(43) Pub. Date: Aug. 8, 2002

(54) Methods for the identification of inhibitors of pyruvate orthophosphate dikinase expression or activity in plants

(76) Inventors: Andreas S. Kloti, Durham, NC (US); Robert A. Ascenzi, Cary, NC (US); Neil E. Hoffman, Chapel Hill, NC (US); Keith R. Davis, Durham, NC (US); Adel Zayed, Durham, NC (US); Jorn Gorlach, Durham, NC (US); Douglas C. Boyes, Chapel Hill, NC (US); Jeffrey P. Woessner, Hillsborough, NC (US); Carol M. Hamilton, Apex, NC (US); Maja C. Kricker, Pittsboro, NC (US)

Correspondence Address: PARADIGM GENETICS, INC, 104 ALEXANDER DRIVE, BUILDING 2, P O BOX 14528, RTP, NC, 27709-4528, US

(21) Appl. No.: 09/734,426

(22) Filed: Dec. 11, 2000

(51) Int. Cl. C12Q 1/48; A01N 25/00; A01N 59/06; A01N 59/14; A01N 59/16; A01N 59/26; A01N 59/02; A01N 59/24; A01N 1/00

(52) U.S. Cl. 435/15; 504/116.1; 504/187; 504/188; 504/189

(57) ABSTRACT

The present inventors have discovered that pyruvate orthophosphate dikinase (PPDK) is essential for plant growth. Specifically, the inhibition PPDK gene expression in plant seedlings results in significant developmental abnormalities, including abnormal cotyledon development, abnormal or aborted primary leaf development and significantly reduced growth. Thus, PPDK can be used as a target for the identification of herbicides. Accordingly, the present invention provides methods for the identification of compounds that inhibit PPDK expression or activity, comprising: contacting a compound with a PPDK and detecting the presence and/or absence of binding between said compound and said a PPDK, or detecting a decrease in PPDK expression or activity. The methods of the invention are useful for the identification of herbicides.

### FIELD OF THE INVENTION

The invention relates generally to plant molecular biology. In particular, the invention relates to methods for the identification of herbicides.

### BACKGROUND OF THE INVENTION

The enzyme pyruvate orthophosphate dikinase (PPDK, EC 2.7.9.1) plays a role in C₄ photosynthesis. Specifically, PPDK catalyzes the reversible conversion of pyruvate to phosphoenolpyruvate. C₄ plants are predomiantly tropical and subtropical. They include maize, millet, sorghum, sugar cane, cordgrass, guinea grass, some Flaveria spp. and Amaranthus spp. Many studies have focused on the role of PPDK in bacteria, protists and C₄ plants. Low levels of this enzyme is have been found in some C₃ and crassulacean acid metabolism (CAM) plants, however the function of this enzyme in C₃ plants is not well characterized. The presence of low levels of PPDK in C₃ plants and in nonphotosynthetic organs of C₄ plants suggests that this enzyme may have a housekeeping function. Glackin et al. (1990) Proc Natl Acad Sci 87:3004-3008; and Rosche et al. (1998) Plant Physiol 117:821-829.

Overexpression of PPDK in plants can result in increased growth and seed production (U.S. Pat. No. 5,891,726, the contents of which is incorporated by reference). However, to date, there has been no report or suggestion that PPDK activity is essential for plant growth and development. Rather, U.S. Pat. No. 5,891,726 teaches that antisense inhibiton of PPDK expression in tobacco, a C₃ plant, has no effect on plant growth or seed production. Thus, the prior art has not suggested that PPDK is a herbicide target.

### SUMMARY OF THE INVENTION

Surprisingly, the present inventors have discovered that antisense expression of a pyruvate, orthophosphate dikinase (PPDK) cDNA in Arabidopsis causes developmental abnormalities, including abnormal cotyledon development, abnormal or aborted primary leaf development and significantly reduced growth of plant seedlings. Thus, the present inventors have discovered that PPDK is essential for normal seed development and growth, and can be used as a target for the identification of herbicides. Accordingly, the present invention provides methods for the identification of compounds that inhibit PPDK expression or activity, comprising: contacting a candidate compound with a PPDK and detecting the presence or absence of binding between said compound and said PPDK, or detecting a decrease in PPDK expression or activity. The methods of the invention are useful for the identification of herbicides.

### BRIEF DESCRIPTION OF THE DRAWINGS

FIG. 1 is a digital image showing the effect of PPDK antisense expression on Arabidopsis thaliana seedlings in two independent pPG949 plant lines.

### DETAILED DESCRIPTION OF THE INVENTION

Definitions

“AMP” is synonomous with “adenosine monophosphate”, “5′-adenylic acid”, and “adenosinephosphoric acid”.

“ATP” is synonymous with “adenosine triphosphate” and “5′-adenyldiphosphoric acid”.

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

The term “herbicide”, as used herein, refers to a compound that may be used to kill or suppress the growth of at least one plant, plant cell, plant tissue or seed.

The term “inhibitor”, as used herein, refers to a chemical substance that inactivates the enzymatic activity of PPDK. The inhibitor may function by interacting directly with the enzyme, a cofactor of the enzyme, the substrate of the enzyme, or any combination thereof.

A polynucleotide may be “introduced” into a plant cell by any means, including transfection, transformation or transduction, electroporation, particle bombardment, agroinfection and the like. The introduced polynucleotide may be maintained in the cell stably if it is incorporated into a non-chromosomal autonomous replicon or integrated into the plant chromosome. Alternatively, the introduced polynucleotide may be present on an extra-chromosomal non-replicating vector and be transiently expressed or transiently active.

The “percent (%) sequence identity” between two polynucleotide or two polypeptide sequences is determined according to the either the BLAST program (Basic Local Alignment Search Tool; Altschul and Gish (1996) Meth Enzymol 266:460-480 and Altschul (1990) J Mol Biol 215:403-410) in the Wisconsin Genetics Software Package (Devererreux et al. (1984) Nucl Acid Res 12:387), Genetics Computer Group (GCG), Madison, Wis. (NCBI, Version 2.0.11, default settings) or using Smith Waterman Alignment (Smith and Waterman (1981) Adv Appl Math 2:482) as incorporated into GeneMatcher Plus™ (Paracel, Inc., http://www.paracel.com/html/genematcher.html; using the default settings and the version current at the time of filing). It is understood that for the purposes of determining sequence identity when comparing a DNA sequence to an RNA sequence, a thymine nucleotide is equivalent to a uracil nucleotide.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “phosphoenolpyruvate” (PEP) is meant a compound of the formula CH₂—COP—COOH or CH₂—COP—COO⁻.

By “polypeptide” is meant a chain of at least four amino acids joined by peptide bonds. The chain may be linear, branched, circular or combinations thereof. The polypeptides may contain amino acid analogs and other modifications, including, but not limited to glycosylated or phosphorylated residues.

By “pyruvate” is meant a compound of the formula CH₃—CO—COOH or CH₃—CO—COO⁻.

As used herein, the term “pyruvate, orthophosphate dikinase” (EC 2.7.9.1) is synonymous with “PPDK”, “ATP:pyruvate, orthophosphate phosphotransferase” and “pyruvate phosphate dikinase”, and refers to an enzyme that catalyses the reversible conversion of pyruvate, ATP and inorganic phosphate (P₁) to phosphoenolpyruvate, AMP and pyrophosphate (PP₁).

The term “specific binding” refers to an interaction between PPDK and a molecule or compound, wherein the interaction is dependent upon the primary amino acid sequence or the conformation of PPDK.

Embodiments of the Invention

The present inventors have discovered that inhibition of gene expression strongly inhibits the growth and development of plant seedlings. Thus, the inventors are the first to demonstrate that pyruvate orthophosphate dikinase is a target for herbicides.

Accordingly, the invention provides methods for identifying compounds that inhibit pyruvate orthophosphate dikinase gene expression or activity. Such methods include ligand binding assays, assays for enzyme activity and assays for pyruvate orthophosphate dikinase gene expression. Any compound that is a ligand for pyruvate orthophosphate dikinase, other than its substrates, pyruvate, ATP and inorganic phosphate or phophoenolpyruvate, AMP and pyrophosphate, or the cofactor zinc may have herbicidal activity. For the purposes of the invention, “ligand” refers to a molecule that will bind to a site on a polypeptide. The compounds identified by the methods of the invention are useful as herbicides.

Thus, in one embodiment, the invention provides a method for identifying a compound as a candidate for a herbicide, comprising:

a) contacting a pyruvate orthophosphate dikinase with said compound; and

b) detecting the presence and/or absence of binding between said compound and said pyruvate orthophosphate dikinase;

wherein binding indicates that said compound is a candidate for a herbicide.

By “pyruvate orthophosphate dikinase” (PPDK) is meant any enzyme that catalyzes the interconversion of pyruvate, ATP and inorganic phosphate to phosphoenolpyruvate, AMP and pyrophosphate. The pyruvate orthophosphate dikinase may have the amino acid sequence of a naturally occuring PPDK found in a plant, animal or microorganism, or may have an amino acid sequence derived from a naturally occuring sequence. Preferably the PPDK is a plant PPDK.

By “plant pyruvate orthophosphate dikinase” is meant an enzyme that can be found in at least one plant, and which catalyzes the interconversion of pyruvate, ATP and inorganic phosphate to phosphoenolpyruvate, AMP and pyrophosphate. The PPDK may be from any plant, including both monocots and dicots, and C₃ plants, C₄ plants and crassulacean acid metabolism (CAM) plants.

In one embodiment, the PPDK is an Arabidopsis PPDK. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. Preferably, the Arabidopsis PPDK is from Arabidopsis thaliana.

The sequences for at A. thaliana PPDK genomic DNAs (gDNA), predicted amino acid sequences and/or cDNAs are known and have been reported at GenBank accession numbers: AL161541 and Z97339 (gDNAs) and A71420, CAB78595, CAB 10331 (SEQ ID NO:1; all identical predicted amino acid sequences). Preferably the PPDK protein is the A. thaliana protein of SEQ ID NO:1.

The PPDK genes and or cDNAs from a variety of organsims are known. See for example, D87745 (Oryza sativa mRNA); X82489 and X78347 (Mesembryanthemum crystallinum); X79192 (Flaveria brownii); X57141 (Flaveria trinervia mRNA); X75516 (C₃ plants mRNA); X16508 and J03901 (maize); BE240866 (Suaeda salsa cDNA); AF194026 (Saccharum officinarum mRNA); D86338 (Elocharis vivipara mRNA); AF079585 (Trypanosoma cruzi PPDK1 and PPDK2); AB025020 (Microbispora rosea); and X74596 (Entamoeba histolytica). All of the above PPDK polynucleotide sequences may be used as probes to isolate PPDK cDNAs or genes from additional organisms, and to synthesize PPDK polypeptides.

In addition, the amino acid sequences of PPDKs from a variety of other plants and organisms are publicly available. See, for example, GenBank accession numbers: S56649 (Flaveria brownii PPDK presursor); S56650 (Flaveria bidentis PPDK precursor); S53297 (Flaveria pringlei); S12894 (Flaveria trinervia PPDK precursor); CAA57872, S55478 and S49497 (Mesembryanthemum crystallinum ); CAA33054, CAA33055 and CAA33056 (maize PPDK1 precursor) and AAA33497 (maize PPDK2); and BAA22420, CAA06247, BAA22419 and T02979 (Oryza sativa ); AAF06668 (Saccharum officinarum); BAA21654 and BAA21653 (Eleocharis vivipara); CAB69782 (Streptomyces coelicolor A3(2)); AAB58820, AAB58819 and AAB58818 (Sinorhizobium meliloti ); AAG12985 (Trypanosoma cruzi); F72397 and AAD35361 (Thermtoga maritima); BAAA76347 (Microbispora rosea); S36601 (Entamoeba histolytica); KIQAPO (Clostridium symbiosum PPDK precursor);.

In various embodiments, the PPDK is from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Fragments of a PPDK polypeptide may be used in the methods of the invention. The fragments comprise at least 10 consecutive amino acids of a PPDK. Preferably, the fragment comprises at least 15, 20, 25, 30, 35, 40, 50, 60, 70, 80, 90 or at least 100 consecutive amino acids residues of a PPDK. In one embodiment, the fragment is from an Arabidopsis PPDK. Preferably, the fragment contains an amino acid sequence conserved among PPDKs. Such conserved fragments are identified in Wei et al. J Biol Chem Sep. 19, 2000 and Pocalyko et al. (1990) Biochemistry 29:10757-10765. Those skilled in the art could identify additional conserved fragments using sequence comparison software. Furthermore, determination of the crystal structure of PPDK has revealed three domains, a phophohistidine domain, a nucleotide domain and a phophoenolpyruvate/pyruvate domain. Herzberg et al. (1996) Proc Natl Acad Sci 93:2652-2657; and Wei et al. (2000), supra. Residues and domains of PPDK that are critical for activity are disclosed by Chastain et al. (2000) Arch Biochem Biophys 375:165-170; McGuire et al. (1998) Biochemistry 37:13463-13474; Chastain et al. (1997) FEBS Lett 413:169-73; McGuire et al. (1996) Biochemistry 35:8544-8552; Yankie et al. (1995) Biochemistry 34:2188-2194; Xu et al. (1995) Biochemistry 34:2195-2202; and Carroll et al. Biochemistry 33:1134-1142.

Polypeptides having at least 80% sequence identity with a plant PPDK are also useful in the methods of the invention. Preferably, the sequence identity is at least 85%, more preferably the identity is at least 90%, most preferably the sequence identity is at least 95%.

In addition, it is preferred that the polypeptide has at least 50% of the activity of a plant PPDK. More preferably, the polypeptide has at least 60%, at least 70%, at least 80% or at least 90% of the activity of a plant PPDK. Most preferably, the polypeptide has at least 50%, at least 60%, at least 70%, at least 80% or at least 90% of the activity of the A. thaliana PPDK protein of SEQ ID NO:1.

Thus, in another embodiment, the invention provides a method for identifying a compound as a candidate for a herbicide, comprising:

a) contacting said compound with at least one polypeptide selected from the group consisting of: a plant PPDK, a polypeptide comprising at least ten consecutive amino acids of a plant PPDK, a polypeptide having at least 85% sequence identity with a plant PPDK, and a polypeptide having at least 80% sequence identity with a plant PPDK and at least 50% of the activity thereof; and

b) detecting the presence and/or absence of binding between said compound and said polypeptide;

wherein binding indicates that said compound is a candidate for a herbicide.

PPDK activity refers to the ability to catalyze the reversible reaction of pyruvate to phosphoenolpyruvate. Both the forward (PEP formation) and the reverse (pyruvate formation) reactions can be measured. Methods for measuring PPDK activity are known in the art. See Ueno et al. (1988) Proc Natl Acad Sci 85:6733-6737; Hiltpold et al. (1999) Mol Biochem Parasitol 104:157-169; and McGuire et al (1998) Biochem 37:13463-13474. For example, both pyruvate and PEP formation can be measured spectrophotometrically at approximately 340 nm via oxidation of NADH in the presence of a coupling system containg lactate dehydrogenase (South et al. (1975) Methods Enzymol 42:187-191) or pyruvate kinase and lactate dehydrogenase (Benzamin (1975) Methods Enzymol 42:192-197), respectively.

Any technique for detecting the binding of a ligand to its target may be used in the methods of the invention. Preferably, the ligand and target are combined in a buffer. In one embodiment, the buffer is 5 mM MgCl₂, 40 mM NH₄Cl and 20 mM imidazole, pH 6.8. In addition, polypeptides and proteins that can reduce non-specific binding, such as BSA, or protein extracts from cells that do not produce the target, may be included in binding assay.

Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with a PPDK protein or a fragment or variant thereof, the unbound protein is removed and the bound PPDK is detected. In a preferred embodiment, bound PPDK is detected using a labeled binding partner, such as a labeled antibody. In a variation of this assay, PPDK is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods. See http://www.evotec.de/technology.

Once a compound is identified as a candidate for a herbicide, it can be tested for the ability to inhibit PPDK enzyme activity. The compounds can be tested using either in vitro or cell based enzyme assays. Alternatively, a compound can be tested by applying it directly to a plant or plant cell, or expressing it therein, and monitoring the plant or plant cell for changes or decreases in growth, development, viability or alterations in gene expression.

Thus, in one embodiment, the invention provides a method for determining whether a compound identified as a herbicide candidate by an above method has herbicidal activity, comprising: contacting a plant or plant cells with said herbicide candidate and detecting the presence or absence of a decrease in the growth or viability of said plant or plant cells.

By decrease in growth, is meant that the herbicide candidate causes at least a 10% decrease in the growth of the plant or plant cells, as compared to the growth of the plants or plant cells in the absence of the herbicide candidate. By a decrease in viability is meant that at least 20% of the plants cells, or portion of the plant contacted with the herbicide candidate are nonviable. Preferably, the growth or viability will be at decreased by at least 40%. More preferably, the growth or viability will be decreased by at least 50%, 75% or at least 90% or more. Methods for measuring plant growth and cell viability are known to those skilled in the art. It is possible that a candidate compound may have herbicidal activity only for certain plants or certain plant species.

The ability of a compound to inhibit PPDK activity can be detected using in vitro enzymatic assays in which the disappearance of a substrate or the appearance of a product is directly or indirectly detected. PPDK catalyzes the reversible reaction of pyruvate, ATP and inorganic phophate to phophoenolpyruvate, AMP and pyrophosphate. Methods for detection of pyruvate, phosphoenolpyruvate, ATP, AMP, inorganic phosphate and pyrophosphate are known to those skilled in the art and include, but are not limited to spectrophotometry, mass spectroscopy, thin layer chromatography (TLC) and reverse phase HPLC.

Thus, the invention provides a method for identifying a compound as a candidate for a herbicide, comprising:

a) contacting pyruvate, ATP and inorganic phosphate with PPDK;

b) contacting said pyruvate, ATP and inorganic phosphate with PPDK and said compound; and

c) determining the concentration of at least one compound selected from the group consisting of pyruvate, ATP, inorganic phosphate, pyruvate, ATP and inorganic phosphate after the contacting of steps (a) and (b).

If a candidate compound inhibits PPDK activity, a higher concentration of the substrates (pyruvate, ATP and inorganic phosphate) and a lower level of the products (phosphoenolpyruvate, AMP and pyrophosphate) will be detected in the presence of the candidate compound (step b) than in the absence of the compound (step a).

Because PPDK is capable of catalyzing both the forward and the reverse reaction, measurement of the conversion of phosphoenolpyruvate, AMP and pyrophosphate to pyruvate, ATP and inorganic phosphate in the presence and absence of a candidate compound can also be used to identify herbicides. Accordingly, in another embodiment, the invention provides a method for identifying a compound as a candidate for a herbicide, comprising:

a) contacting phosphoenolpyruvate, AMP and pyrophosphate with PPDK;

b) contacting said phosphoenolpyruvate, AMP and pyrophosphate with PPDK and said compound; and

c) determining the concentration of at least one compound selected from the group consisting of phosphoenolpyruvate, AMP, pyrophosphate, pyruvate, ATP and inorganic phosphate after the contacting of steps (a) and (b).

In this case, if a candidate compound inhibits PPDK activity, a higher concentration of the substrates (phosphoenolpyruvate, AMP and pyrophosphate) and a lower level of the products (pyruvate, ATP and inorganic phosphate) will be detected in the presence of the candidate compound (step b) than in the absence of the compound (step a).

Preferably the PPDK is a plant PPDK. Enzymatically active fragments of a plant PPDK are also useful in the methods of the invention. For example, a polypeptide comprising at least 100 consecutive amino acid residues of a plant PPDK may be used in the methods of the invention. In addition, a polypeptide having at least 80%, 85%, 90%, 95%, 98% or at least 99% sequence identity with a plant PPDK may be used in the methods of the invention. Preferably, the polypeptide has at least 80% sequence identity with a plant PPDK and at least 50%, 75%, 90% or at least 95% of the activity thereof.

Thus, the invention provides a method for identifying a compound as a candidate for a herbicide, comprising:

a) contacting pyruvate, ATP and inorganic phosphate with a polypeptide selected from the group consisting of: a polypeptide having at least 85% sequence identity with a plant PPDK, a polypeptide having at least 80% sequence identity with a plant PPDK and at least 50% of the activity thereof, and a polypeptide comprising at least 100 consecutive amino acids of a plant PPDK;

b) contacting said pyruvate, ATP and inorganic phosphate with said polypeptide and said compound; and

c) determining the concentration of at least one compound selected from the group consisting of pyruvate, ATP, inorganic phosphate, phosphoenolpyruvate, AMP and pyrophosphate after the contacting of steps (a) and (b).

Again, if a candidate compound inhibits PPDK activity, a higher concentration of the substrate (pyruvate, ATP and inorganic phosphate) and a lower level of the product (phosphoenolpyruvate, AMP and pyrophosphate) will be detected in the presence of the candidate compound (step b) than in the absence of the compound (step a).

In an alternative embodiment, the invention provides a method for identifying a compound as a candidate for a herbicide, comprising:

a) contacting phosphoenolpyruvate, AMP and pyrophosphate with a polypeptide selected from the group consisting of: a polypeptide having at least 85% sequence identity with a plant PPDK, a polypeptide having at least 80% sequence identity with a plant PPDK and at least 50% of the activity thereof, and a polypeptide comprising at least 100 consecutive amino acids of a plant PPDK;

b) contacting phosphoenolpyruvate, AMP and pyrophosphate with said polypeptide and said compound; and

c) determining the concentration of at least one of at least one compound selected from the group consisting of: phosphoenolpyruvate, AMP, pyrophosphate, pyruvate, ATP and inorganic phosphate after the contacting of steps (a) and (b).

In this case, if a candidate compound inhibits PPDK activity, a higher concentration of the substrates (phosphoenolpyruvate, AMP and pyrophosphate) and a lower level of the products (pyruvate, ATP and inorganic phosphate) will be detected in the presence of the candidate compound (step b) than in the absence of the compound (step a).

For the in vitro enzymatic assays, PPDK protein and derivatives thereof may be purified from a plant or may be recombinantly produced in and purified from a plant, bacteria, or eukaryotic cell culture. Preferably these proteins are produced using a baculovirus or E. coli expression system. Methods for the purification of PPDK are described in U.S. Pat. No. 6,054,305, the contents of which is incorporated by reference; Bringaud et al. (1998) Proc Natl Acad Sci 95:7963-7968; Hiltpold et al. (1999) Mol Biochem Parasitol 104:157-159 and Eisaki et al. (1999) Biochem Biophys Acta 1431:363-373. Other methods for the purification of PPDK proteins and polypeptides are known to those skilled in the art.

As an alternative to in vitro assays, the invention also provides plant and plant cell based assays. In one embodiment, the invention provides a method for identifying a compound as a candidate for a herbicide, comprising:

a) measuring the expression of PPDK in a plant or plant cell in the absence of said compound;

b) contacting a plant or plant cell with said compound and measuring the expression of PPDK in said plant or plant cell;

c) comparing the expression of PPDK in steps (a) and (b).

A reduction in PPDK expression indicates that the compound is a herbicide candidate. In one embodiment, the plant or plant cell is an Arabidopsis thaliana plant or plant cell. Preferably the A. thaliana PPDK is the PPDK of SEQ ID NO:1.

Expression of PPDK can be measured by detecting PPDK primary transcript or mRNA, PPDK polypeptide or PPDK enzymatic activity. Methods for detecting the expression of RNA and proteins are known to those skilled in the art. See, for example, Current Protocols in Molecular Biology Ausubel et al., eds., Greene Publishing and Wiley-Interscience, New York, 1995. The method of detection is not critical to the invention. Methods for detecting PPDK RNA include, but are not limited to amplification assays such as quantitative PCR, and/or hybridization assays such as Northern analysis, dot blots, slot blots, in-situ hybridization, transcriptional fusions using a PPDK promoter fused to a reporter gene, bDNA assays and microarray assays.

Methods for detecting protein expression include, but are not limited to, immunodetection methods such as Western blots, His Tag and ELISA assays, polyacrylamide gel electrophoresis, mass spectroscopy and enzymatic assays. Also, any reporter gene system may be used to detect PPDK protein expression. For detection using gene reporter systems, a polynucleotide encoding a reporter protein is fused in frame with PPDK, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2:1104; Prost et al. (1986) Gene 45:107-111), β-galactosidase (Nolan et al. (1988) Proc Natl Acad Sci USA 85:2603-2607), alkaline phosphatase (Berger et al. (1988) Gene 66:10), luciferase (De Wet et al. (1987) Mol Cell Biol 7:725-737), β-glucuronidase (GUS), fluorescent proteins, chromogenic proteins and the like. Methods for detecting PPDK activity are described above.

Chemicals, compounds or compositions identified by the above methods as modulators of PPDK expression or activity can then be used to control plant growth. For example, compounds that inhibit plant growth can be applied to a plant or expressed in a plant, in order to prevent plant growth. Thus, the invention provides a method for inhibiting plant growth, comprising contacting a plant with a compound identified by the methods of the invention as having herbicidal activity.

Herbicides and herbicide candidates identified by the methods of the invention can be used to control the growth of undesired plants, including both monocots, dicots, C₃ and C₄ plants and CAM plants. Examples of C₃ plants include, but are not limited to Arabidopsis, rice and tobacco. Examples of C₄ plants include, but are not limited to maize, millet, sorghum, sugar cane, cordgrass, guinea grass, Flaveria trinervia, Flaveria brownii and Amaranthus spp.

Examples of undesired plants include, but are not limited to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### Experimental

Plant Growth Conditions

Unless, otherwise indicated, all plants were grown Scotts Metro-Mix™ soil (the Scotts Company) or a similar soil mixture in an environmental growth room at 22° C., 65% humidity, 65% humidity and a light intensity of ˜100 μ-E m⁻² s⁻¹ supplied over 16 hour day period.

Seed Sterilization

All seeds were surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dl H₂O; seeds should be stirred up by pipetting of water into tube. Carefully remove water. Repeat 3 to 5 times to ensure removal of Clorox/SDS solution.

7. Fill each tube with enough sterile dl H₂0 for seed plating (˜200-400 μl). Cap tube until ready to begin seed plating.

Plate Growth Assays

Surface sterilized seeds were sown onto plate containing 40 ml half strength sterile MS (Murashige and Skoog, no sucrose) medium and 1% Phytagel using the following protocol:

1. Using pipette man and 200 μl tip, carefully fill tip with seeds and 0.1% agarose solution. Place 10 seeds across the top of the plate, about ¼ in down from the top edge of the plate.

2. Place plate lid ¾ of the way over the plate and allow to dry for 30 minutes or until agarose solution is dry. It is important to allow agarose solution to dry completely before sealing up plates in order to prevent contamination.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-E m⁻² s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### EXAMPLE 1

### Construction of a Transgenic Plant Expressing the Driver

The “Driver” is an artificial transcription factor comprising a chimera of the DNA-binding domain of the yeast GAL4 protein (amino acid residues 147) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### EXAMPLE 2

### Construction of PPDK Antisense Expression Cassettes in a Binary Vector

A fragment of an Arabidopsis thaliana PPDK cDNA shwon in SEQ ID NO:2 and corresponding to GenBank accession No. 7434943 was ligated into the PacI/AscI sites of the E.coli/Agrobacterium binary vector PGT3.2 in the antisense orientation. This placed transcription of the PPDK antisense RNA under the control of an artificial promoter that is active only in the presence of the driver transcription factor described above. The artificial promoter contains four contiguous binding sites for the GAL4 transcriptional activator upstream of a minimal promoter comprising a TATA box.

The ligated DNA was transformed into E. coli. Kanamycin resistant clones were selected and purified. DNA was isolated from each clone and characterized by PCR and sequence analysis. pPG949 expresses the A. thaliana PPDK antisense RNA encoded by the DNA of SEQ ID NO:2.

The antisense expression cassette and a constitutive barnase expression cassette are located between right and left T-DNA borders. Thus, the antisense expression cassettes can be transferred into a recipient plant cell by agroinfection.

### EXAMPLE 3

### Transformation of Agrobacterium with the Target Expression Cassette

pPG949 was transformed into Agrobacterium tumefaciens by electroporation. Transformed Agrobacterium colonies were isolated using Basta selection. DNA was prepared from purified Basta resistant colonies and the inserts were amplified by PCR and sequenced to confirm sequence and orientation.

### EXAMPLE 4

### Construction of an Arabidopsis PPDK Antisense Target Plants

The PPDK target expression cassette was introduced into Arabidopsis thaliana wild-type plants by the following method. Five days prior to agroinfection, the primary inflorescence of Arabidopsis thaliana plants grown in 2.5 inch pots were clipped in order enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying pPG237 or pPG244. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L Silwet L-77™ (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then cover with a tall clear plastic dome in order to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected using glufosinate treatment. Approximately 70 mg seeds from an agrotransformed plant were mixed approximately 4:1 with sand and placed in a 2 ml screw cap cryo vial.

The surface of the seeds was sterilized using the chlorine gas method. Briefly, the open vials were placed in a vacuum desiccator in a safety hood. A glass beaker containing 200 ml 5.25% sodium hypochlorite solution was placed in the desiccator.

Two ml concentrated HCl was added to the hypochlorite solution and the cover was placed on the desiccator. Vacuum was applied briefly to seal the dessicator, and the seeds were left in the desiccator overnight.

One vial of sterilized seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a 1:3000 dilution of LibertyTM (AgrEvo; 11.3% glufosinate) in water, 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. These herbicide resistant plants represent stably transformed T1 plants. Mature T1 plants are then dried and harvested for T2 seeds.

### EXAMPLE 5

### Effect of PPDK Antisense Expression in Arabidopsis Seedlings

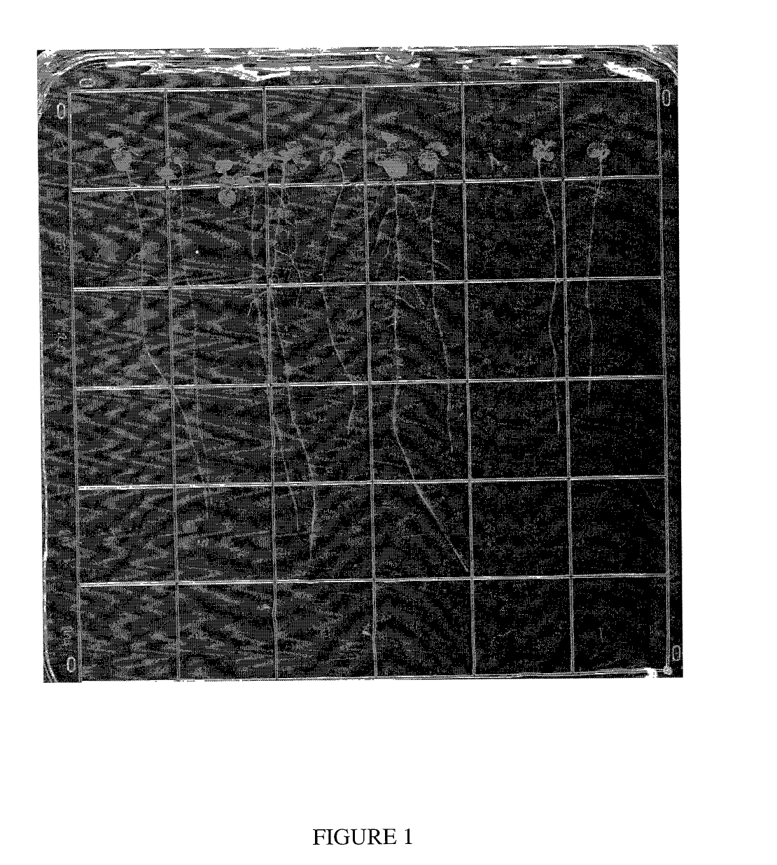

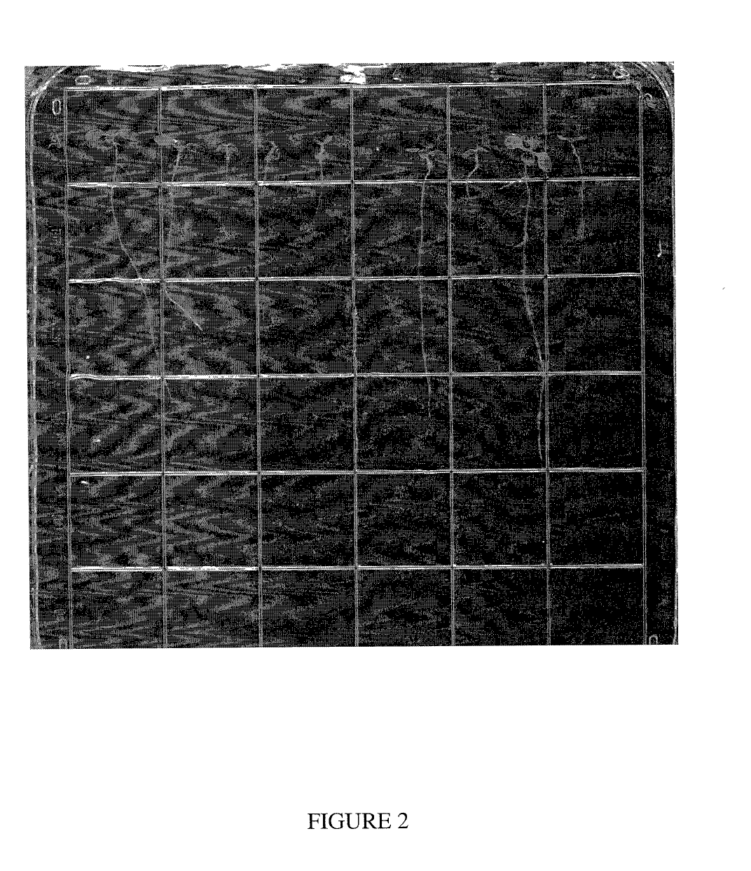

The PPDK target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected daily for growth and development. During this period, approximately half of seedlings derived from two independent pPG949 PPDK antisense target lines exhibited significant developmental abnormalotoes, including abnormal cotyledon development, abnormal or aborted primary leaf development and significantly reduced growth. FIG. 1 shows the effect of PPDK antisense expression on Arabidopsis seedlings. The results are summarized in Table 1.

TABLE 1
Phenotypes of plants expressing PPDK antisense RNA

| Construct | No. Wild Type | No. Abnormal | χ² Value^{a} | Probability^{a} |
|---|---|---|---|---|
| pPG949 | 4 | 5 | 0.111 | 0.739 |
| Line 1 | | | | |
| Line 2 | 6 | 4 | 0.400 | 0.527 |

^{a}Chi-square and P values (0.05) were obtained to evaluate the hypothesis that chlorosis and wild-type phenotypes are segregating in a 1:1 ratio.

The clear 1:1 segregation ration observed in two independent antisense lines expressing pPG949 demonstrates that the antisense expression of PPDK results in significantly impaired growth. Thus, PPDK is essential for normal plant growth and development.

### EXAMPLE 6

### Assay for Inhibitors of PPDK Activity

The enzymatic activity of PPDK is determined by a spectrophotometric assay in the presence and absence of candidate inhibitors in a reaction mixture containing 0.5 mM AMP, 0.5 mM PEP, 1 mM PP₁ 5 mM MgCl₂, 40 mM NH₄Cl and 20 mM imidazole, pH 6.8 and recombinant PPDK enzyme at 25° C.

While the foregoing describes certain embodiments of the invention, it will be understood by those skilled in the art that variations and modifications may be made and still fall within the scope of the invention.

1. A method for identifying a compound as a candidate for a herbicide, comprising:
a) contacting a pyruvate orthophosphate dikinase with said compound; and
b) detecting the presence and/or absence of binding between said compound and said pyruvate orthophosphate dikinase;
wherein binding indicates that said compound is a candidate for a herbicide.

2. The method of claim 1, wherein said pyruvate orthophosphate dikinase is a plant pyruvate orthophosphate dikinase.

3. The method of claim 2, wherein said pyruvate orthophosphate dikinase is an Arabidopsis pyruvate orthophosphate dikinase.

4. The method of claim 3, wherein said pyruvate orthophosphate dikinase is the Arabidopsis thaliana protein of SEQ ID NO:1.

5. A method for determining whether a compound identified as a herbicide candidate by the method of claim 1 has herbicidal activity, comprising: contacting a plant or plant cells with said herbicide candidate and detecting the presence or absence of a decrease in growth or viability of said plant or plant cells.

6. A method for identifying a compound as a candidate for a herbicide, comprising:
a) contacting said compound with at least one polypeptide selected from the group consisting of: an amino acid sequence comprising at least ten consecutive amino acids of a plant pyruvate orthophosphate dikinase, an amino acid sequence having at least 85% sequence identity with a plant pyruvate orthophosphate dikinase, and an amino acid sequence having at least 80% sequence identity with a plant pyruvate orthophosphate dikinase and at least 50% of the activity thereof, and
b) detecting the presence and/or absence of binding between said compound and said polypeptide;
wherein binding indicates that said compound is a candidate for a herbicide.

7. A method for determining whether a compound identified as a herbicide candidate by the method of claim 6 has herbicidal activity, comprising: contacting a plant or plant cells with said herbicide candidate and detecting the presence or absence of a decrease in growth or viability of said plant or plant cells.

8. A method for identifying a compound as a candidate for a herbicide, comprising:
a) contacting pyruvate, ATP and inorganic phosphate with PPDK;
b) contacting said pyruvate, ATP and inorganic phosphate with PPDK and said compound; and
c) determining the concentration of at least one compound selected from the group consisting of pyruvate, ATP, inorganic phosphate, pyruvate, ATP and inorganic phosphate after the contacting of steps (a) and (b).

9. The method of claim 8, wherein said pyruvate orthophosphate dikinase is a plant pyruvate orthophosphate dikinase.

10. The method of claim 9, wherein said pyruvate orthophosphate dikinase is an Arabidopsis pyruvate orthophosphate dikinase.

11. A method for identifying a compound as a candidate for a herbicide, comprising:
a) contacting phosphoenolpyruvate, AMP and pyrophosphate with PPDK;
b) contacting said phosphoenolpyruvate, AMP and pyrophosphate with PPDK and said compound; and
c) determining the concentration of at least one compound selected from the group consisting of phosphoenolpyruvate, AMP, pyrophosphate, pyruvate, ATP and inorganic phosphate after the contacting of steps (a) and (b).

12. The method of claim 11, wherein said pyruvate orthophosphate dikinase is a plant pyruvate orthophosphate dikinase.

13. The method of claim 12, wherein said pyruvate orthophosphate dikinase is an Arabidopsis pyruvate orthophosphate dikinase.

14. A method for identifying a compound as a candidate for a herbicide, comprising:
a) contacting pyruvate, ATP and inorganic phosphate with a polypeptide selected from the group consisting of: a polypeptide having at least 85% sequence identity with a plant PPDK, a polypeptide having at least 80% sequence identity with a plant PPDK and at least 50% of the activity thereof, and a polypeptide comprising at least 100 consecutive amino acids of a plant PPDK;
b) contacting said pyruvate, ATP and inorganic phosphate with said polypeptide and said compound; and
c) determining the concentration of at least one compound selected from the group consisting of pyruvate, ATP, inorganic phosphate, phosphoenolpyruvate, AMP and pyrophosphate after the contacting of steps (a) and (b).

15. A method for identifying a compound as a candidate for a herbicide, comprising:
a) contacting phosphoenolpyruvate, AMP and pyrophosphate with a polypeptide selected from the group consisting of: a polypeptide having at least 85% sequence identity with a plant PPDK, a polypeptide having at least 80% sequence identity with a plant PPDK and at least 50% of the activity thereof, and a polypeptide comprising at least 100 consecutive amino acids of a plant PPDK;
b) contacting phosphoenolpyruvate, AMP and pyrophosphate with said polypeptide and said compound; and
c) determining the concentration of at least one of at least one compound selected from the group consisting of: phosphoenolpyruvate, AMP, pyrophosphate, pyruvate, ATP and inorganic phosphate after the contacting of steps (a) and (b).

16. A method for identifying a compound as a candidate for a herbicide, comprising:
a) measuring the expression of a pyruvate orthophosphate dikinase in a plant or plant cell in the absence of said compound;
b) contacting a plant or plant cell with said compound and measuring the expression of said pyruvate orthophosphate dikinase in said plant or plant cell;
c) comparing the expression of pyruvate orthophosphate dikinase in steps (a) and (b).

17. The method of claim 16 wherein said plant or plant cell is an Arabidopsis plant or plant cell.

18. The method of claim 16, wherein said pyruvate orthophosphate dikinase is the pyruvate orthophosphate dikinase protein of SEQ ID NO:1.

19. The method of claim 16, wherein the expression of pyruvate orthophosphate dikinase is measured by detecting pyruvate orthophosphate dikinase mRNA.

20. The method of claim 16, wherein the expression of pyruvate orthophosphate dikinase is measured by detecting pyruvate orthophosphate dikinase polypeptide.
